(19) United States
(12) Patent Application Publication
(10) Pub. No.: US 2008/0312587 A1
(43) Pub. Date: Dec. 18, 2008

(54) System for the Oral Administration of Solids to Persons Suffering from Dementia

(75) Inventors: Heinrich Kugelmann, Aachen (DE); Johannes Bartholomaus, Aachen (DE); Eric-Paul Paques, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: GRUENETHAL GMBH, AACHEN (DE)

(21) Appl. No.: 11/917,098

(22) PCT Filed: Jun. 7, 2005
(86) PCT No.: PCT/EP2006/005412

§ 371 Date: Jul. 15, 2008

(30) Foreign Application Priority Data
Jun. 10, 2005 (DE) 10 2005 027 051.4

(51) Int. Cl. A61M 37/00

(52) U.S. Cl. 604/84

(57) ABSTRACT

The invention relates to a system for the oral administration of a solid to dementia sufferers comprising at least two devices, in which each device comprises an optionally transparent hollow article with a liquid inlet end and a liquid outlet end, wherein a solid and a liquid-permeable, optionally displaceable retention means are arranged in the interior of the hollow article in such a manner that the retention means prevents the solid from escaping from the interior of the hollow article via the liquid inlet end.

The invention relates to a system for the oral administration of solids, in particular of nutritional supplements and medicaments, to persons suffering from dementia.

Dementia is a general term used to describe the inability of people to make use of their own mental abilities. Dementia as a consequence of brain disease generally has a chronic or progressive course with impairment of many higher cortical functions, including memory, thought, orientation, comprehension, calculation, learning capacity, language and judgement. Cognitive impairment is generally accompanied by impairment of emotional control, social behaviour and motivation.

According to ICD-10, the WHO classification system, dementia is accompanied by memory disorders, degradation of the capacity to think, personality changes, distinct impairment of independence in daily life and a duration of symptoms exceeding 6 months.

Irrespective of the primary cause, all different forms of dementia seem be accompanied by neuronal dysfunction. This dysfunction is characterised by disruption of neurotransmission in the brain, with declining synthesis or reduced release of various neurotransmitters or reduced post-receptor action playing a variable role depending on the type of disease.

The clinical process of cognitive degradation progresses slowly and affects most middle-aged and elderly people. The following three stages in the disease are generally recognised:

1st stage: Short-term memory is impaired. Patients carry out tasks again which have already been completed, repeat themselves and repeatedly question the same things. Date and time can no longer be stated. Complicated interrelationships cause increasing difficulties. Affected individuals behave more passively and indifferently, which may eventually amount to apathy.
2nd stage: Patients now already require greater assistance from other people. It is no longer possible to carry out an occupation and daily life is considerably limited. Memory difficulties increase. Simple daily tasks such as personal hygiene, getting dressed and eating require ever greater effort and, ultimately, can only be carried out with outside help. Patients can hardly find their way around in their own home. Past and present times become confused. A declining sense of taste results in loss of appetite.
3rd stage: Patients are now completely dependent on outside help and care. Contact can now only be established with difficulty. Psychological symptoms are now also accompanied by a loss of physical control, with bladder and bowel control being lost.

In recent times, various highly promising therapeutic options have been developed for dementia, including new psychopharmaceuticals which are capable of slowing the progression of the disease or even bringing it to a complete standstill. These pharmaceutical substances, which are conventionally also known as “antidementia agents”, are usually provided as solid dosage forms, such as for example film coated tablets.

However, oral administration of antidementia agents to dementia patients causes numerous difficulties which may be of various kinds depending on the stage to which the disease has progressed in the patient.

Many dementia patients accordingly have difficulties even at an early stage of their disease with taking tablets, gelatine capsules, etc., since they frequently have a tendency to choke during taking, even if or precisely because they take the solid dosage form together with a lot of liquid. As a result, dementia patients sometimes develop a strong aversion to these solid dosage forms.

One conceivable alternative to tablets which may in principle be considered is liquid dosage forms, but these are frequently characterised by lower stability of the pharmaceutical substance, pharmacokinetic properties which can be influenced only with difficulty and sometimes an unpleasant odour or bitter flavour. Furthermore it is difficult to provide an accurate dosage, since the medicament is not conventionally divided into precisely defined, individually packaged units to be taken individually, but is instead provided in a storage container from which the patient in each case takes the necessary dose, for example with the assistance of a teaspoon to measure volume or by counting out drops from a suitable dispenser. Such liquid dosage forms are unsuitable for dementia patients even at the early stage of the disease who are not usually under the constant supervision of care staff, since measuring the dose of the medicament should not be the responsibility of the dementia sufferer. Due to the nature of the disease, there would otherwise be a considerable risk of over- or underdosing the medicament and continuous medication thus could not be ensured.

Certain prior art solid dosage forms, for example for oral administration of antidementia agents, sometimes require adjustment of the dosage by the patient, for example if for therapeutic reasons a specific dosage regimen must be observed over an extended period. For example, the recommended daily dose of memantine hydrochloride, which is used for the treatment of Alzheimer's disease in the form of film coated tablets with a pharmaceutical substance content of 10 mg, is 20 mg for adults. In order to reduce the risk of side-effects, this dose is reached progressively in accordance with a treatment plan. The treatment plan accordingly begins by administering half a film coated tablet in the morning in the first week of treatment, administering half a film coated tablet in each case in the morning and either in the afternoon or in the evening in the second week of treatment, administering a whole film coated tablet in the morning and half a film coated tablet either in the afternoon or in the evening in the third week of treatment and administering a whole film coated tablet in each case in the morning and either in the afternoon or in the evening in the fourth week of treatment (details according to AXURA® patient information leaflet, 08/2002).

This comparatively complicated dosage regimen, which also entails breaking the film coated tablet, cannot be followed independently even by patients with dementia at an early stage of the disease, such that compliance with such a dosage regimen usually has to be entrusted to care staff or the patient's next-of-kin.

Supervision by the next-of-kin or care staff may in principle be achieved in various ways. At a stage in the disease which is not yet too far advanced, it is usually sufficient for the member of care staff to visit the dementia sufferer once daily in his/her home.

However, even when administering a constant dose of the pharmaceutical substance, there is always a risk with dementia sufferers, due to the nature of the disease, of its being forgotten to take an individual dosage unit. This may result in over- or underdosage which, on the one hand, may jeopardise therapeutic success but, on the other hand, may also involve serious health risks. Accordingly, even checking of the administration of the medicament by care staff, who establish, for example once daily, whether the number of film coated tablets to be taken for the corresponding day have been pressed out of a blister pack, is not capable of effectively minimising the risk of the patient's forgetting to take the medicament. This type of checking carries with it the not inconsiderable risk for dementia sufferers that, while the patient has indeed pressed the film coated tablet out of the blister pack over the course of the day, he/she has subsequently forgotten to take it and has instead, for example, put the film coated tablet down somewhere else in his/her home.

This risk could be effectively avoided if the dementia sufferer were to take the film coated tablet in the presence of the care staff. However, since numerous medicaments, including antidementia agents, usually have to be taken several times daily, this would be associated with considerable expenditure even for patients suffering from an early stage of dementia.

Automatic monitoring systems have been developed which permit checking of whether or not a medicament has been administered to a patient at a specific time. DE-A 101 23 720, for example, accordingly discloses a method for monitoring medicament-assisted therapy of patients with reduced cognitive and reactive abilities (for example dementia). The doctor can be automatically notified if the patient makes a mistake in taking the medicament. This method does, however, involve a relatively high level of technical complexity.

There are additional difficulties with dementia patients at an advanced stage of the disease. Accordingly, it is frequently difficult even for trained specialist staff to persuade dementia sufferers to take the medicament at all, even if the attending member of staff monitors administration at the time of taking and offers the patient appropriate assistance. Dementia patients at an advanced stage of the disease accordingly often behave passively and apathetically and do not respond to the care staff's request to take a dosage form. Even if a film coated tablet is placed in the patient's mouth by care staff, it is possible for the tablet to remain in the oral cavity because the patient does not actively assist with swallowing.

The object of the invention is accordingly to provide a system for the oral administration of medicaments or nutritional supplements to dementia sufferers which exhibits advantages relative to the prior art. The system is intended to provide dementia sufferers with assistance in self-checking the taking of the required, optionally varying dose of the physiologically active substance (pharmaceutical substance, vitamin, trace element, etc.) over an extended period and to reduce the risk of forgetting, due to the nature of the disease, to take individual dosage units. Subsequent reliable checking should also be possible, for example by care staff. The system should furthermore make it easier for dementia sufferers to take the solids orally; in particular, oral administration should also be possible without the conscious assistance of the patient. The system should preferably enable reliable oral administration of medicaments or nutritional supplements by patients at all stages of dementia.

It has surprisingly been found that this object may be achieved by a system comprising at least two devices, in which each device comprises an optionally transparent hollow article with a liquid inlet end and a liquid outlet end, wherein a solid and a liquid-permeable, optionally displaceable retention means are arranged in the interior of the hollow article in such a manner that the retention means prevents the solid from escaping from the interior of the hollow article via the liquid inlet end.

Simple embodiments of such and similar devices are known in the prior art. Reference may be made in this connection, for example, to EP-A 383 503 and EP-A 840 591. However, systems comprising two or more such devices are hitherto unknown.

The system according to the invention comprises at least two such devices. The system according to the invention preferably comprises a plurality of such devices, preferably 3, 4, 5, 6, 7, 8, 10, 14, 15, 20, 21, 25, 28, 30, 31, 40, 42, 50, 56, 61, 63, 64, 75, 84, 100, 112, 365, 730 or 1095 devices.

The individual devices in the system according to the invention contain a solid. Virtually all substances which may advantageously be administered orally to a dementia sufferer, preferably foodstuffs and nutritional supplements, in particular also medicaments, may be considered as the solid.

Nutritional supplements preferably contain one or more nutrients in a concentrated, dosed form which is atypical of foodstuffs. They are intended to supplement daily food intake in those cases in which the amount supplied by foods is inadequate or supplementation is desired. Examples of nutritional supplement are vitamins, minerals, trace elements, enzymes, fatty acids, amino acids, antioxidants, hormones, etc.

Medicaments are preferably substances or preparations of substances which are intended by the manufacturer or the party marketing them to be used for therapeutic or diagnostic purposes. Medicaments contain at least one pharmaceutical substance (active ingredient) which is the reason for which the medicament is used and is the cause of the efficacy thereof. A medicament may additionally contain conventional auxiliary materials, such as fillers, binders, slip agents, disintegrants, flow-control agents, surfactants, emulsifiers, antioxidants, aromas, coatings, etc.

The solid is preferably present in the devices of the system according to the invention in the form of particles, pellets, granules or microcapsules. The average particle diameter is here preferably in the range from 25 to 5,000 μm, more preferably 50 to 2,500 μm, still more preferably 75 to 1,000 μm and in particular 100 to 500 μm.

In a preferred embodiment of the system according to the invention, the at least two devices are identically or differently labelled, preferably with numbers and/or letters. Suitable labels are, in particular, parts of the day (e.g. morning, midday, afternoon, evening, nighttime), times, names of days of the week or their abbreviations and names of months or their abbreviations.

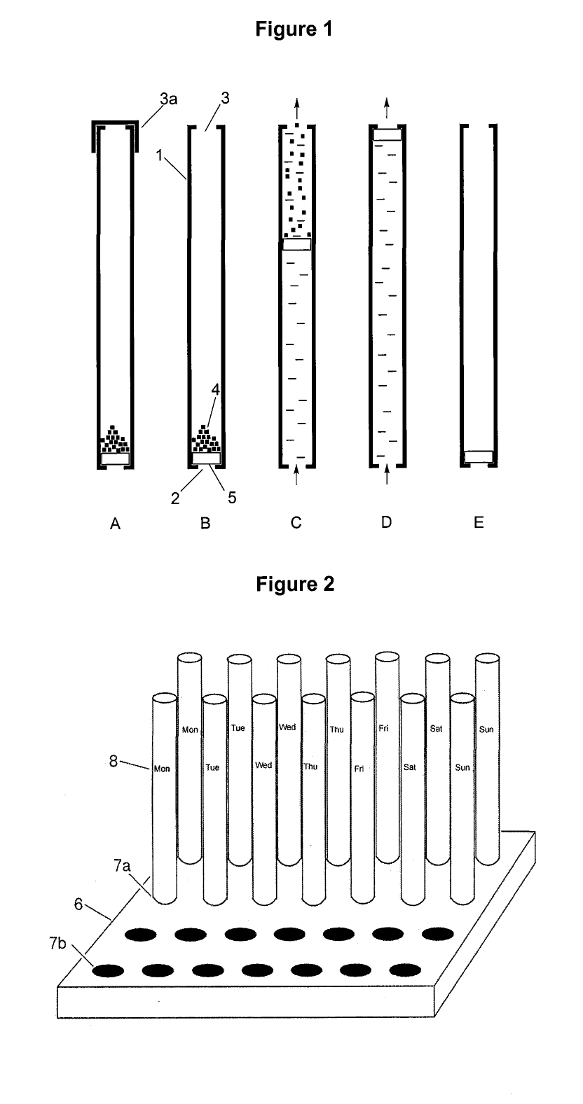

The system according to the invention is preferably used as shown schematically in FIGS. 1A to E. An individual device is first taken from the system (FIG. 1A). An optionally present protective cap (3a) is then removed from the liquid outlet end (3) (FIG. 1B) and the liquid inlet end (2) is dipped into a physiologically acceptable liquid (for example water, a beverage, soup etc.). The patient then puts his/her mouth to the liquid outlet end and sucks up the liquid through the device, in a similar way as with a drinking straw. As a result of the suction action, the liquid penetrates the liquid-permeable retention means (5) and swirls up the solid (4) located between the liquid inlet end (2) and the liquid outlet end (3), such that said solid is supplied in dispersed form together with the liquid to the patient's mouth and escapes from the interior of the hollow article (1) via the liquid outlet end (3) (FIG. 1 C). After use, the device is either discarded or returned to the system according to the invention.

The retention means (5) is preferably displaceable between the liquid inlet end (2) and the liquid outlet end (3), such that, as a result of the flow action of the liquid sucked in, it moves contrary to gravity in the direction of the liquid outlet end (3) and so allows the patient to check whether all the solid (4) has already been removed from the device or whether still more liquid must be sucked in. If the hollow article (1) is in fact made from a transparent material, the patient can see at any time the exact position of the retention means (5). Once all the solid (4) has been removed from the device, the retention means (5) is prevented from passing into the patient's mouth by a suitable stop means at the liquid outlet end (3) (FIG. 1D). When the patient stops sucking up liquid, the retention means (5) preferably drops back down under the force of gravity to its initial position (FIG. 1E).

The liquid-permeable retention means (5) is preferably made from a material which contains orifices which allow a liquid which has been sucked up to pass through but which retain the solid. Suitable materials are known to a person skilled in the art. Coarse-mesh fabrics, filters etc., preferably of plastics, for example of polyethylene are, for example, suitable.

The system according to the invention has the advantage that it enables checking of whether the dementia sufferer has or has not already taken a specific individual dose. In the initial stage of his/her disease, a dementia sufferer can still him/herself carry out this check. Simply looking at the system according to the invention makes it possible to check how many of the originally present at least two devices have already been removed and how many are still available for future administration. Unlike in the case tablets which are for example taken from a storage jar, in the system according to the invention the empty device is left behind after oral administration of the solid contained in a specific device and this can be an aid to the dementia sufferer's memory. In contrast, finding out whether a tablet has or has not already been removed from the storage container is only possible by recounting the tablets still present in the container.

According to the invention, the system is preferably designed such that it can reaccommodate the individual devices after they have been removed and used by the dementia sufferers. This is preferably achieved by the system comprising a frame which comprises at least one holder for each one of the at least two devices. The holders may here be configured such that they both release the devices for removal and also reaccommodate them after use. This may be achieved, for example, by a plate which contains holes with a diameter which is somewhat larger than the diameter of the devices. The devices may then be placed with their lower end, conventionally the liquid inlet end, in the holes. In this manner, each of the devices has its own place on the frame predetermined by the holder, so facilitating checking.

In a preferred embodiment, the system according to the invention comprises a frame which comprises two spatially separate holders a and b for each one of the at least two devices. The ready-to-use devices are at the beginning all located in the type a holders, such that the type b holders are initially empty. After a patient has used a device, he/she returns the emptied device, if the system is used correctly, to the system in the predetermined type b holder.

In the example shown in FIG. 2, two groups of devices labelled with abbreviations of the days of the week (8) are positioned in type a holders. This arrangement preferably corresponds to the arrangement of the system according to the invention in the unused state. The front row of devices is for oral administration of a solid in the morning (unit dose), while the back row is for administration of the solid in the evening (unit dose). On Monday morning, the dementia sufferer (or the member of care staff) takes the device located in holder (7a) and uses it as instructed, i.e. administers the solid contained therein to him/herself. He/she now does not return the used device to position (7a), but instead places it in position (7b). On Monday evening, he/she repeats this procedure similarly with the device located in the back row etc.

In order to enable unambiguous differentiation between type a and type b holders, the type a holders are preferably designed such that, while they do indeed allow the device to be taken out, they do not reaccommodate it after use. The only option open to the patient for placing the device back into the system after use is therefore a type b holder.

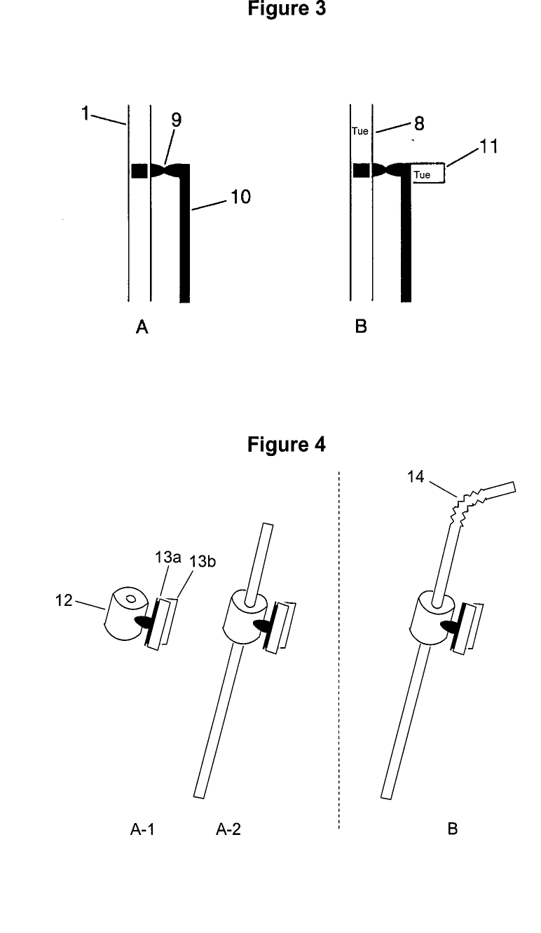

One possible way in which such type a holders may be designed is shown in FIG. 3 A. The individual devices are fastened onto plastics rods (10), which are firmly connected at one end to the frame and are fastened to the device at the other end, for example by means of a necked contact point (9). The necked portion (9) weakens the mechanical stability of the plastics material at this point in such a manner that, by simple hand movements with application of only very slight force, the device can be moved to and fro relative to the frame, whereby the plastics material breaks or tears at the necked point such that the device is finally released. The type b holders for accommodating the devices after they have been used may, for example, be configured as holes in a plate.

In a preferred embodiment, the type a and/or type b holders and/or the devices are identically or differently labelled with numbers and/or letters, as FIG. 3 B shows schematically for an example type a holder.

In a preferred embodiment of the system according to the invention, the at least two devices may be classified into at least two groups. Various properties of the devices may be used as the criterion for classifying the devices in this manner.

In a preferred embodiment, devices of the same group contain the solid in the same quantity, while devices of different groups contain the solid in a different quantity. Such a system makes it possible to implement even complicated dosage regimens, as are for example recommended for the pharmaceutical substance memantine hydrochloride. Accordingly, devices of one group may contain the pharmaceutical substance in a unit dose of 5 mg and devices of another group may contain the pharmaceutical substance in a unit dose of 10 mg.

In another preferred embodiment, devices of the same group have the same size and/or colour, while devices of different groups have a different size and/or colour. This makes it easier for the dementia sufferer to make a purposeful selection of a specific device. Additional labelling may enhance this advantage still further.

The individual groups of devices are preferably arranged spatially separate from one another within the system.

For example, for twice daily administration of memantine hydrochloride over a period of one month in compliance with a specific dosage regimen, it is possible to provide seven or eight groups, each group comprising seven devices. The devices of the first group have a specific colour, size and/or labelling (for example “week 1—morning”) and contain the pharmaceutical substance in a unit dose of 5 mg. An optional second group of another colour, size and/or labelling (for example “week 1—evening”) contains no pharmaceutical substance. The third and fourth group, in each case of another colour, size and/or labelling (for example “week 2—morning”/“week 2—evening”), in turn contain the pharmaceutical substance in a unit dose of 5 mg. The fifth group of another colour, size and/or labelling (for example “week 3—morning”) contains the pharmaceutical substance in a unit dose of 10 mg, etc.

In this manner, it is possible to ensure controlled oral administration of the pharmaceutical substance at different dosages, with reliable assistance being offered to the dementia sufferer (and also care staff) in selecting, at the particular time, the correct device with the correct dosage from within the system. In this manner, it is possible completely to dispense with independent dose measurement, as is for example required when breaking film coated tablets into two parts.

The system according to the invention may alternatively also be provided as a container with lid, wherein the lid may be flipped open and is connected to the container by hinges.

In a preferred embodiment of the system according to the invention, the at least two devices are arranged in a frame which is equipped with visual and/or acoustic medication taking time indicators. Light-emitting diodes affixed to the frame which provide a reminder to use a specific device at a specific time by lighting up may, for example, be used for this purpose. A suitable tone generator may optionally emit an alarm signal at the same time as the LED lights up. A preferred embodiment provides that the luminous and/or acoustic alarm signal does not cease until the corresponding device has been taken out of the frame. If, in addition to the above-described type a holders, type b holders are also provided which serve to accommodate the used device after the use thereof, the luminous and/or acoustic alarm signal preferably does not cease according to the invention until the corresponding device has been taken from the type a holder and placed back in the type b holder.

The solid in the devices of the system according to the invention preferably comprises a medicament. In principle, any medicaments which may meaningfully be administered orally to dementia sufferers may be considered.

Examples of pharmaceutical substances (active ingredients) contained in the medicaments which may be mentioned are: adrenoceptor agonists, adrenoceptor antagonists, sympathomimetics, antisympathomimetics, muscarine receptor agonists, muscarine receptor antagonists, nicotine receptor agonists, nicotine receptor antagonists, cholinesterase inhibitors, analgesics, hypnotics, convulsive drugs, antiepileptics, central muscle relaxants, antiparkinsons drugs, psychopharmaceuticals, antidementia agents, inflammation and allergy mediators, antiarrhythmic agents, positive ionotropic substances, cardiac glycosides, phosphodiesterase inhibitors, vasodilatators, diuretics, aldosterone antagonists, anticoagulants, coagulation inhibitors, fibrinolysis inhibitors, iron, emetics, antiemetics, laxatives, uricosurics, uricostatics, lipid-reducing agents, orally active blood glucose reducing substances, hormones, geriatric drugs, antibiotics, chemotherapeutic agents, virostatic agents, immunosuppressants, etc. Reference is made to the entire disclosure of the following documents with regard to further details and subgroups: K. Aktories et al., Aligemeine und spezielle Pharmakologie und Toxikologie, Urban & Fischer, November 2004; and E. Mutschler et al., Arzneimittelwirkungen, Wissenschaftliche Verlagsgesellschaft, January 2001.

In addition to the above-stated pharmaceutical substances, the medicaments may contain conventional auxiliary materials for solid dosage forms, such as for example fillers, binders, slip agents, disintegrants, flow-control agents, surfactants, emulsifiers, antioxidants, aromas, enteric coatings, etc. With regard to further details, reference is made to the entire disclosure of H. P. Fiedler, Lexikon derHilfsstoffe für Pharmazie, Kosmetik und angrenzende Gebiete, 2 volumes, Editio Cantor Aulendorff, January 2002.

The medicaments assume solid form. They preferably comprise powders, granules, tablets, coated solid dosage forms or capsules of suitable size, for example also micro- or nanoparticles. With regard to further details, reference is made to the entire disclosure of K. H. Bauer et al., Lehrbuch derpharmazeutischen Technologie, Wissenschaftliche Verlagsgesellschaft, 1999.

In a preferred embodiment of the system according to the invention, the at least two devices contain the medicament in a total quantity which corresponds to a daily, weekly, monthly, quarterly, six monthly or annual dose of the pharmaceutical substance for a dementia sufferer contained in the medicament.

The medicament preferably contains an antidementia agent as the pharmaceutical substance. The antidementia agent is particularly preferably selected from the group consisting of acetylcholinesterase inhibitors, NMDA receptor antagonists and the pharmaceutically acceptable salts thereof. The antidementia agent is preferably selected from the group consisting of tacrine (Cognex®), donepezil (Aricept®), galantamine (Reminyl®), rivastigmine (Exelon®), dizocilpine (MK 801), neramexane (MRZ 2/579), dextrophan, dextromethorphan, ketamine, memantine (Axura®, Namenda®), amantadine, felbamate, acamprosate, phencyclidine and aptiganel, or the pharmaceutically acceptable salts thereof. Salts which may be considered are, for example, hydrochlorides, citrates, maleates, fumarates, acetates, propionates, etc. The antidementia agent particularly preferably comprises 3,5-dimethyl-9-aminoadamantane (memantine) or one of the pharmaceutically acceptable salts thereof, in particular the hydrochloride thereof (memantine hydrochloride).

A further aspect of the invention relates to a device for the oral administration of antidementia agents to dementia sufferers, which device comprises an optionally transparent hollow article with a liquid inlet end and a liquid outlet end, wherein an antidementia agent in solid form and a liquid-permeable, optionally displaceable retention means are arranged in the interior of the hollow article in such a manner that the retention means prevents the antidementia agent in solid form from escaping from the interior of the hollow article via the liquid inlet end.

The antidementia agent is preferably selected from the group consisting of acetylcholinesterase inhibitors, NMDA receptor antagonists and the pharmaceutically acceptable salts thereof. The antidementia agent is particularly preferably selected from the group consisting of tacrine (Cognex®), donepezil (Aricept®), galantamine (Reminyl®), rivastigmine (Exelon®), dizocilpine (MK 801), neramexane (MRZ 2/579), dextrophan, dextromethorphan, ketamine, memantine (Axura®, Namenda®), amantadine, felbamate, acamprosate, phencyclidine and aptiganel, or the pharmaceutically acceptable salts thereof.

The antidementia agent particularly preferably comprises 3,5-dimethyl-9-aminoadamantane (memantine) or one of the pharmaceutically acceptable salts thereof, in particular the hydrochloride thereof (memantine hydrochloride).

Preferred embodiments of this device according to the invention are described above in connection with the system according to the invention (for example size, colour, labelling etc.).

A further aspect of the invention relates to a device for the oral administration of solids to dementia sufferers, which device can be fastened to the patient's clothing. This device comprises an optionally transparent hollow article with a liquid inlet end and a liquid outlet end, wherein a solid, preferably an antidementia agent as described above, and a liquid-permeable, optionally displaceable retention means are arranged in the interior of the hollow article in such a manner that the retention means prevents the solid from escaping from the interior of the hollow article via the liquid inlet end, and wherein the device is equipped with a fastening means which, in itself or by cooperating with an auxiliary fastening means, is suitable for fastening the device to a patient's clothing. The fastening means and/or auxiliary fastening means preferably comprises a safety pin or a spring clip.

FIG. 4 shows an example of device according to the invention comprising a fastening means and auxiliary fastening means. FIG. 4 A-1 shows an auxiliary fastening means (12) with a spring-loaded clip, the clip comprising two jaws (13a, 13b) by means of which the auxiliary fastening means may be fastened to the dementia sufferer's clothing. The device according to the invention may be inserted into an opening in the auxiliary fastening means (12) and held in place with the assistance of a suitable mechanism (FIG. 4 A-2). FIG. 4 B shows a preferred embodiment in which a portion of the wall of the hollow article is pleated in such a manner that bending is possible in order to make it easier for the dementia sufferer to suck up the liquid.

Further preferred embodiments of this device according to the invention are described above in connection with the system according to the invention (for example size, colour, labelling etc.).

These embodiments have the advantage that the patient can carry the device with him/herself during the day and is so constantly reminded of taking the solid contained therein.

A further aspect of the invention relates to a device for the oral administration of solids to dementia sufferers, which device comprises a mouthpiece. This device comprises an optionally transparent hollow article with a liquid inlet end and a liquid outlet end, wherein a solid, preferably an antidementia agent as defined above, and a liquid-permeable, optionally displaceable retention means are arranged in the interior of the hollow article in such a manner that the retention means prevents the solid from escaping from the interior of the hollow article via the liquid inlet end, wherein the mouthpiece
on the one hand prevents the solid from trickling out from the interior of the hollow article when the device is oriented in such manner that a gravitational force acts on the solid in the direction of the mouthpiece, and
on the other hand permits escape of the solid via the liquid outlet end of the device when a stream of liquid is passed from the liquid inlet end to the liquid outlet end.

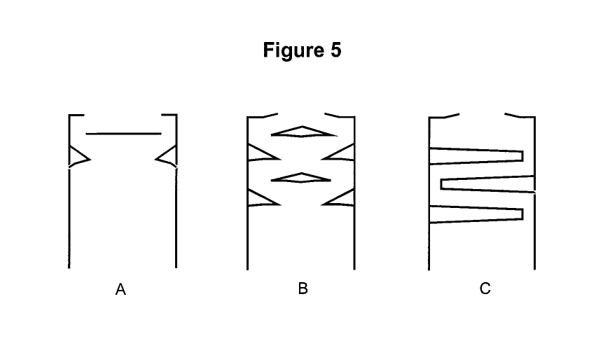

FIGS. 5 A to C are schematic diagrams of preferred embodiments of such a mouthpiece. Further possible ways in which the desired function may be implemented are known to a person skilled in the art.

Preferred embodiments of this device according to the invention are described above in connection with the system according to the invention and the device which may be fastened to the dementia sufferer's clothing (for example size, colour, labelling etc.).

In comparison with film coated tablets, which are for example provided in blister packs, this embodiment of the device according to the invention offers the additional advantage that virtually the only way in which the solid can be removed from the device is by means of a stream of liquid which has been sucked in. Removal of the solid without its simultaneously being taken by the patient is scarcely possible, whereby an emptied device, unlike an emptied blister pack, is a reliable indication for care staff that the dementia sufferer has actually taken the medicament. In the event that the hollow article is transparent, it is possible to establish with the naked eye whether solid still remains in the device (=administration has not occurred) or whether the device has already been emptied (=administration has occurred).

An advantage of this embodiment is furthermore that the device may also still be used if it has, for example, carelessly been dropped on the floor, since in this case the solid cannot simply escape from the interior of the hollow article. Furthermore, there is also no risk in the case of proper use of the device of the solid escaping unintentionally from the hollow article via the liquid outlet end. This may for example be of significance if the patient cannot hold the device still in his/her hand due to Parkinson's disease which occasionally accompanies dementia.

The devices according to the invention are preferably made from a transparent material so that an observer can establish visually whether the device does or does not contain solid and so that, if the retention means is displaceable, an observer can identify the current position of the retention means within the hollow article. The hollow article preferably takes the form of a tube which preferably has a cross-section of 2 to 10 mm, more preferably of 3 to 8 mm, and a length of preferably 10 to 30 cm. Reference may furthermore be made to EP-A 840 591 with regard to further developments and preferred materials.

It has surprisingly been found that, when used correctly, the devices according to the invention make it easier for dementia sufferers to take solids to be administered orally, such as for example medicaments and nutritional supplements. In this manner, oral administration of solids to dementia sufferers is sometimes possible even at an advanced stage of the disease without their conscious assistance.

Due to the nature of the disease, it is thus frequently necessary, in particular for elderly patients, to provide food in chopped, pureed or mushy form since chewing meat, vegetables and fruit causes difficulties. Dementia sufferers have thus frequently already become used over an extended period, often for some years, to consuming food and drink by means of aids, such as for example drinking straws. While administering tablets seems somewhat unusual and strange to these patients, the oral administration of solids with the assistance of the devices according to the invention causes fewer difficulties because such devices can fit unnoticed into the patient's normal daily routine. Some dementia sufferers clearly associate the necessity of consuming food and drink with such aids, for example with drinking straws. In this way, said association may successfully be exploited by the device according to the invention to encourage a dementia sufferer to take medicaments, in particular also antidementia agents, because, in contrast to the administration of a tablet, he/she is used to drinking straws and will assume that they are simply intended for the intake of food and drink.

A further aspect of the invention relates to the use of one of the above-described devices according to the invention for producing an above-described system for the treatment or prevention of dementia, in particular for the treatment or prevention of
dementia arising from processes of brain atrophy selected from the group consisting of
   Alzheimer-type dementia,
   Pick's disease,
   Lewy body dementia,
   frontal lobe type dementia,
   Huntington's chorea,
   corticodentatonigral degeneration,
   progressive supranuclear opthalmoplegia,
   pallidum degeneration,
   spinocerebellar degeneration,
   myoclonic epilepsies, or
   Hallervorden-Spatz disease; or
of secondary forms of dementia arising from causes selected from the group consisting of
   hydrocephalus aresorptivus,
   cardiovascular diseases (extracranial: cardiac insufficiency, cardiac infarct; intracranial: arteriosclerotic stenoses, hypertensive encephalopathy, congophilic angiopathy, angiitides, arteriovenous malformations, Binswanger's disease, subcortical arteriosclerotic encephalopathy),
   haematological diseases,
   respiratory disorders (chronic and respiratory insufficiency),
   metabolic diseases and avitaminoses (diabetes mellitus including hypo- and hyperglycaemia, Addison's disease, Cushing's disease, hypo- and hyperthyroidism, hypo- and hyperparathyroidism, Fahr's syndrome, electrolyte imbalance including central pontine myelinolysis, lipid storage diseases and glycogen storage diseases, mitochondrial cytopathies, Wilson's disease, nephrogenic encephalopathy, dialysis encephalopathy, hepatogenic encephalopathy, porphyria, malnutrition, malabsorption and avitaminoses of vitamins B₁, B₆, B₁₂, folic acid and nicotinic acid),
   poisoning (alcoholism, carbon monoxide, heavy metals, organic compounds),
   infectious, parainfectious and immunological diseases (meningoencephalitides (viral, bacterial, mycotic), lues, brain abscess, disseminated encephalomyelitis (multiple sclerosis), Creutzfeldt-Jakob disease, HIV, progressive multifocal leucoencephalopathy, Gerstmann-Sträussler disease), tumours (brain tumour, lymphomas, haematological malignomas and paraneoplastic diseases),
   trauma (brain contusion, subdural haematoma),
   chronic subdural haematoma (subdural hygroma and condition following subarachnoid bleeding),
   epilepsies and
   psychiatric diseases.

The following examples serve to illustrate the invention in greater detail but should not to be construed to limit the scope thereof:

### EXAMPLE 1

a) 10 drill holes with a diameter of 10 mm were drilled in a block of polymethyl methacrylate. 5 drill holes were initially arranged in a row and numbered consecutively from 1 to 5. A second row of five identically numbered drill holes was arranged parallel thereto. The first row was additionally labelled “return”, while the second row was labelled “removal position”.

b) A 20 cm long tube of polyethylene with a diameter of 8 mm was carefully narrowed to a diameter of approx. 6 mm by heating at one end. The length of the narrowed portion was approx. 4 mm. An approx. 5 mm tall cylinder of fine-pore polyethylene sponge with a diameter of approx. 8 mm was introduced into the tube through the non-narrowed end of the tube and pushed along to the narrowed end. 25.5 mg of tacrine hydrochloride monohydrate were weighed out and introduced into the tube via the non-narrowed end. The other end of the tube was then also narrowed to a diameter of approx. 6 mm by heating and the end narrowed in this manner was closed with a plastics cap.

A total of five tubes were produced by this method and were labelled with the numbers 1 to 5.

The labelled tubes were inserted into the correspondingly numbered removal positions in the stand.

### EXAMPLE 2

a) A stand with drill holes was produced in a similar manner to Example 1a). The stand contained 6 drill holes in two rows each of 3 holes, the three removal positions additionally being labelled with the removal times “8:00”, “12:00” and “18:00” respectively.

b) In a manner similar to Example 1b), 3 tubes were each filled with a dose of 7.5 mg of memantine hydrochloride and inserted into the three removal positions on the stand. The pharmaceutical substance memantine hydrochloride was here provided in film coated pellets which, in addition to the pharmaceutical substance, contained lactose monohydrate, microcrystalline cellulose, highly disperse silicon dioxide, talcum and magnesium stearate as auxiliary substances, and were coated with a film of methacrylic acid/ethyl acrylate copolymer (1:1), sodium dodecyl sulfate, polysorbate 80, talcum, triacetin and simethicone emulsion.

1-21. (canceled)

22. A system for orally administering a solid to a dementia sufferer, said system comprising at least two devices, wherein each device comprises:
a hollow article with a liquid inlet end and a liquid outlet end,
a solid to be administered disposed within said hollow article, and
a liquid-permeable retainer arranged in the interior of the hollow article such that the retainer prevents the solid from escaping the interior of the hollow article via the liquid inlet end.

23. A system as claimed in claim 22, wherein said hollow article is transparent.

24. A system as claimed in claim 22, wherein said retainer is displaceable within said hollow article.

25. A system as claimed in claim 22, wherein the at least two devices labelled with identifying characters.

26. A system as claimed in claim 25, wherein different devices are labelled with different identifying characters.

27. A system as claimed in claim 26, wherein the different identifying characters are each indicative of an intended timepoint of administration.

28. A system as claimed in claim 22, wherein the at least two devices are classified into at least two groups, wherein the devices in each group contain the same quantity of said solid, and the devices of different groups contain different quantities of said solid.

29. A system as claimed in claim 28, wherein the groups are arranged spatially separate from one another within the system.

30. A system as claimed in claim 22, wherein the at least two devices are classified into at least two groups, wherein the devices in each group have the same size or colour or both, and the devices of different groups differ in size or color or both.

31. A system as claimed in claim 30, wherein the groups are arranged spatially separate from one another within the system.

32. A system as claimed in claim 22, further comprising a frame which contains two spatially separate holders for each of the at least two devices.

33. A system as claimed in claim 32, wherein said separate holders are each labelled with identifying characters.

34. A system as claimed in claim 22, wherein said solid is a medicament.

35. A system as claimed in claim 34, wherein the total quantity of the medicament contained in the at least two devices corresponds to a daily, weekly, monthly, quarterly, six monthly or annual dose for the dementia sufferer.

36. A system as claimed in claim 34, wherein said medicament comprises an antidementia agent.

37. A system as claimed in claim 36, wherein said antidementia agent is selected from the group consisting of acetylcholinesterase inhibitors, NMDA receptor antagonists and pharmaceutically acceptable salts thereof.

38. A system as claimed in claim 36, wherein said antidementia agent is selected from the group consisting of tacrine, donepezil, galantamine, rivastigmine, MK 801, MRZ 2/579, dextrophan, dextromethorphan, ketamine, memantine, amantadine, felbamate, acamprosate, phencyclidine, aptiganel and pharmaceutically acceptable salts thereof.

39. A device for orally administering an antidementia agent to a dementia sufferer comprising a hollow article with a liquid inlet end and a liquid outlet end, an antidementia agent within said hollow article, and a liquid-permeable retainer arranged in the interior of the hollow article such that the retainer prevents the antidementia agent from escaping the interior of the hollow article via the liquid inlet end.

40. A device as claimed in claim 39, wherein said hollow article is transparent.

41. A device as claimed in claim 39, wherein said retainer is displaceable within said hollow article.

42. A device as claimed in claim 39, wherein the antidementia agent is selected from the group consisting of acetylcholinesterase inhibitors, NMDA receptor antagonists, and pharmaceutically acceptable salts thereof.

43. A device as claimed in claim 39, wherein the antidementia agent is selected from the group consisting of tacrine, donepezil, galantamine, rivastigmine, MK 801, MRZ 2/579, dextrophan, dextromethorphan, ketamine, memantine, amantadine, felbamate, acamprosate, phencyclidine, aptiganel, and pharmaceutically acceptable salts thereof.

44. A device as claimed in claim 39, further comprising a fastener for securing said device to a patient's clothing.

45. A device as claimed in claim 44, wherein said device cooperates with an auxiliary fastener.

46. A device as claimed in claim 44, wherein said fastener comprises a safety pin or a spring clip.

47. A device as claimed in claim 39, further comprising a mouthpiece at said liquid outlet end, said mouthpiece preventing the antidementia agent from escaping the interior of the hollow article when the device is oriented such that a gravitational force acts on the antidementia agent in the direction of the mouthpiece, and said mouthpiece permitting escape of the antidementia agent via the liquid outlet end of the device when a stream of liquid is passed through the hollow article from the liquid inlet end to the liquid outlet end.

48. A method of treating or inhibiting dementia arising from processes of brain atrophy selected from the group consisting of Alzheimer's disease, Pick's disease, Lewy body dementia, frontal lobe type dementia, Huntington's chorea, corticodentatonigral degeneration, progressive supranuclear opthalmoplegia, pallidum degeneration, spinocerebellar degeneration, myoclonic epilepsies and Hallervorden-Spatz disease; or of secondary forms of dementia arising from causes selected from the group consisting of hydrocephalus aresorptivus, cardiovascular diseases, haematological diseases, respiratory disorders, metabolic diseases and avitaminoses, poisoning, infectious, parainfectious and immunological diseases, tumours, trauma, chronic subdural haematoma, epilepsies and psychiatric diseases in a subject in need thereof, said method comprising administering to said subject a pharmacologically effective amount of an antidementia agent by causing said subject to ingest a liquid through a device as claimed in claim 39.
